(19) United States
(12) Patent Application Publication
(10) Pub. No.: US 2005/0063964 A1
(43) Pub. Date: Mar. 24, 2005

(54) Yeast-based curative and care product and method for making same

(76) Inventor: Gabriele Wolf, Isernhagen (DE)

Correspondence Address: JACOBSON HOLMAN PLLC, 400 SEVENTH STREET N.W., SUITE 600, WASHINGTON, DC, 20004, US

(21) Appl. No.: 10/498,516

(22) PCT Filed: Dec. 19, 2002
(86) PCT No.: PCT/EP02/14543

(30) Foreign Application Priority Data
Dec. 21, 2001 (EP) 01130634.7

(51) Int. Cl. C12P 1/04; A61K 38/43

(52) U.S. Cl. 424/94.1; 424/93.45; 435/170

(57) ABSTRACT

A method for the preparation of a curative and care agent for the skin on the basis of an acidified leaven, wherein: 1.1 a natural enzymatic starter and water are added to a rye fine or coarse meal, the mixture is subjected to a process of heating to 65-75° C. within 3 to 5 hours, a strong maltose formation being initiated from enzymatic reactions; 1.2 a further addition of rye fine or coarse meal, water and a bacteriological inoculum from the group of heterofermentative lactic-acid bacteria is made; and 1.3 the mixture is acidified until the metabolic activity of the microorganisms ceases. The invention further relates to a curative and care agent for the skin obtainable by said method, and to the use of said skin and care agent.

The invention relates to a method for the preparation of a curative and care agent for the skin based on an acidified leaven, a curative and care agent obtainable by said method, a cosmetic preparation containing a curative and care agent, and the use of the curative and care agent according to the invention in a cosmetic preparation.

From DE-A-38 02 840 2, a method for the preparation of a fermentation product containing lactic acid and viable lactic-acid bacteria by acidifying an aqueous slurry of a baked leaven-based bread mass in a lactic acid containing medium has already been known, the baked product of an acidified bread dough being employed as the bread mass. This fermentation product is said to be employable, inter alia, as an auxiliary agent in the treatment of the holistic organism of humans, animals and plants.

From DE-A-38 46 186, it has also been known already to employ preparations obtained from the fermentation sludge of spontaneous acidification of leaven breads, for example, as a pack aggregate material for the treatment of chronic inflammatory diseases of the skin.

From DT-B-26 11 972, it has already been known to acidify the leaven bacteria in the cereal mash forming the pre-dough until any bacteriological metabolic activity ceases for the preparation of a leaven for the preparation of breads and bakery products from cereal coarse and/or fine meals.

In EP-B-0 530 861, a topical antimicrobial pharmaceutical composition which comprises a mixture of C₆ to C₁₈ fatty acids has been disclosed.

DE-U-299 23 627 relates to a curative and care agent for promoting the blood flow, for releasing muscular tension, for skin cleaning and for skin regeneration, which is applied to body parts or used as an additive to a water bath. The product is characterized by being formed from a fermentation product of an acidified leaven. This document does not mention anything about a pretreatment of the rye coarse meal used as a substrate.

WO-A-00/10395 discloses a method for the preparation of a live liquid leaven product which has a plaque-forming capacity of at least 5×10⁸ cfu/g of meal (dry matter) after cold storage for 10 weeks. No indications are given as to the fact that this product is suitable as a curative and care agent. In this document, calcium carbonate is employed as a buffer agent in order that the pH value of the mixture described therein does not decrease too much.

Natural products formed by fermentation which are brought into contact with the skin have a potential of positively influencing skin which is in need of care.

It was the object of the present invention to improve the positive influence, especially the effects known from DE-U-299 23 627.

Surprisingly, fermentation products of rye coarse or fine meals with heterofermentative lactic-acid bacteria, which have been treated with a-amylase prior to fermentation, are suitable for achieving the object of the invention.

The present invention relates to a method for the preparation of a curative and care agent for the skin from fermentation products based on an acidified leaven, wherein:
1.1 a natural enzymatic starter and water are added to a rye fine or coarse meal, the mixture is subjected to a process of heating to 65-75° C. within 3 to 5 hours, a strong maltose formation being initiated from enzymatic reactions;
1.2 a further addition of rye fine or coarse meal, water and a bacteriological inoculum from the group of heterofermentative lactic-acid bacteria is made; and
1.3 the mixture is acidified to a pH value of <4 until the metabolic activity of the microorganisms ceases.

In particular, α-amylase is employed as an enzymatic starter.

In a particular embodiment of the method according to the invention, Lactobacillus DSM 6037 and/or Lactobacillus DSM 6129 are preferably employed.

In a further embodiment of the method according to the invention, a second carbohydrate-containing substrate and water as well as a bacteriological inoculum from the group of heterofermentative lactic-acid bacteria are added to the fermentation product, and the mixture is acidified;
the latter process step is optionally repeated once or several times, and the mixture is finally acidified until any bacteriological metabolic activity ceases; and
the fermentation product is subjected to a stabilizing heating process at near to the boiling temperature.

The acidification is preferably performed to a pH value of 3.3-3.5.

The invention relates to a curative and care agent for the skin based on an acidified leaven and obtainable by the method according to the invention.

The curative and care agent according to the invention advantageously has a high content of lactic acid obtained in a fermentative way of >60% by weight (as a fraction of the total amount of acid).

The curatives and care agents according to the invention can be admixed with auxiliary agents and carriers usual in the cosmetic art to prepare cosmetic preparations.

The curatives and care agents according to the invention which can be employed are used, in particular, in the form of powders, rye fluids, rye sera, rye gels.

The cosmetic preparation according to the invention which can be prepared from the curatives and care agents according to the invention can be employed, in particular, as an active ingredient at from 1 to 100% by weight, especially from 2 to 50% by weight, based on the total amount, in ointments, powders, creams, lotions, gels, shampoos, hair conditioners, packs, lipsticks, face and body masks, or combinations thereof.

Thus, for example, a face mask for cleaning can be prepared from ingredients usual in cosmetic formulations, such as steareth-12 or steareth-21, glyceryl stearates, stearic acid, alkyl benzoates with chain lengths, in particular, of from 12 to 15 carbon atoms, cetearyl isononaoate, stearyl heptanoate, octyl stearate, fragrants or flavors, such as Persea gratissima avocado oil (supplied by Cosmo-Care), the rye gel or rye fluid according to the invention, propylene glycol, fillers, such as china clay and water. Another application form can predominantly consist of serum according to the invention having a high lactic acid content, as well as vitamins and other supplements. A shampoo may typically contain ingredients such as sodium laureth sulfate, disodium laureth sulfosuccinate, cocoamidopropyl betaine, PEG-7 glyceryl cocoate, the rye serum according to the invention, fillers such as styrene/acrylate copolymers/laureth-23, citric acid, polyquaternium-10 (supplied by Amerchol), PEG-18 glyceryl oleate or cocoate, as well as water, preservatives and fragrances. A typical curing pack may consist of cetearyl alcohol, ceteareth-12, ceteareth-20, PEG-75 lanolin, Triticum vulgare (supplied by Dragoco), the rye fluid or rye serum according to the invention, hydroxycetyl or hydroxyethyl dimonium chloride (supplied by Henkel), panthenol, citric acid, as well as water, preservatives and fragrances.

A hair conditioner may consist, in particular, of the rye fluid or rye serum according to the invention with fragrances, preservatives and fillers, for example, fruit vinegar, glycerol, cetearyl alcohol, ceteareth-12, cetrimonium chloride, PEG-75 lanolin, preservatives, fragrances and water. The ingredients mentioned are chemicals usually employed in the cosmetic art and are all commercially available.

In one embodiment of the present invention, the curative and care agent according to the invention is employed in the form of a rye fluid. It is a creamy raw material obtained from rye gel which is excellently suitable for creams, lotions, shampoos, hair conditioners and packs.

The rye gel according to the invention is a rather high-viscous raw material which may also be employed directly as a curative and care agent according to the invention. It is the product which has more or less immediately been obtained from the fermentation process, which can be employed unchanged as a natural face or body mask and as a bath additive. An advantage of this embodiment is the lacking of any components which are possibly not tolerated and put a burden on the organism. After the application, improvements of the skin relief and a deceleration of the formation of skin impurities are found. The agent may serve as a biological peeling agent for died skin scales, in which a moisturizing effect advantageously appears.

From rye fluid, a rye serum can in turn be obtained. It is suitable as a raw material to be incorporated in face and body masks, lipsticks, hair shampoos and hair packs.

Starting from rye gel, for example, rye powder, rye fluid and rye serum can be prepared. Starting from rye gel, the rye powder can be prepared by separating the solids from the rye gel according to the invention, which is subsequently dried and ground. The solids from the rye powder can be employed in the cosmetic preparations according to the invention as peeling bodies. For example, the rye fluid is prepared by separation from the purified gel and enrichment with unfiltered rye serum until the viscosity suitable for use has been adjusted. This product is a raw material having a high content of active substance. The rye serum is prepared, for example, by filtering the liquid supernatant of the rye gel according to the invention obtained from the fermentation, followed by clarification. This yields a product having a high content of active ingredient.

The invention also relates to the use of the curatives and care agents according to the invention in a cosmetic preparation according to the invention.

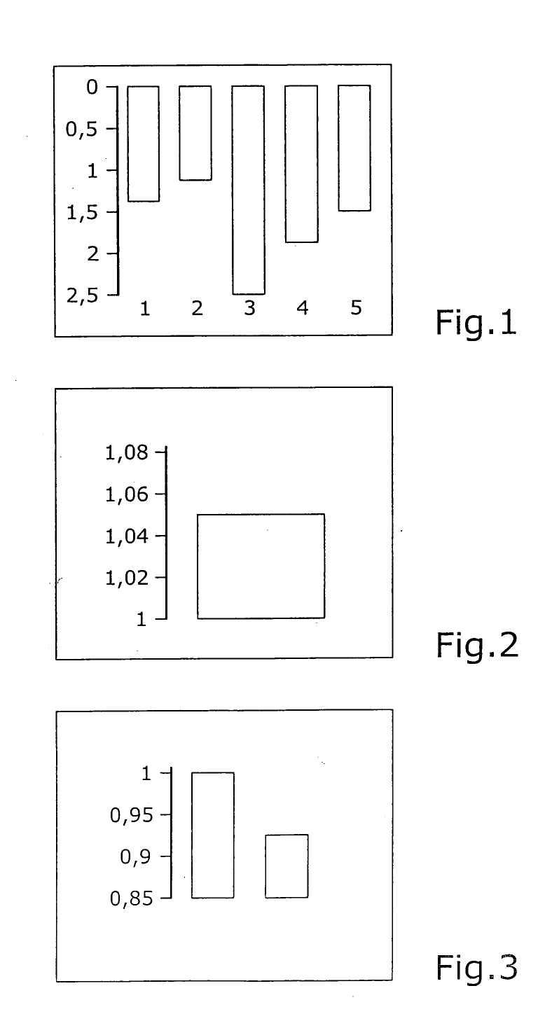

FIG. 1 shows the influence of a treatment with a rye gel mask on the appearance of impure skin. The reduction of the lesions in subjects 1 to 5 was by a factor of up to 2.5.

FIG. 2 illustrates the moisturizing effect. The increase of skin moisture is shown.

FIG. 3 relates to skin microtopography and illustrates the reduction of skin roughness in two subjects.

### EXAMPLE 1

To prepare the rye gel according to the invention, one part of rye meal 1150 is mixed with two parts of water, and a starter culture with a-amylase is added. The mixture is heated at 65 to 75° C. for 3 to 5 hours. After the start of the enzymatic reaction, the starting fermentation is initiated by mixing one part of rye meal 1150 with two parts of water. To this mixture, fermentation media with Lactobacillus DSM 6037 and Lactobacillus DSM 6129 are added. This mixture is fermented for 24 to 48 hours at temperatures of from 30 to 34° C. The fermentation is complete when a constant pH value establishes. The mixture obtained from the starting fermentation is admixed with 60 parts of water, based on the total volume of the starting fermentation. The fermentation time is 24 to 48 hours at a typical temperature of from 30 to 34° C. until a constant pH value has been reached. This is followed by a thermal treatment for 3 to 5 hours at a temperature of from 92 to 100° C. Optionally, preservatives, such as sorbic acid and benzoic acid, are added. Typically, 0.75 parts of sorbic acid can be employed, based on the total volume. The preservation step is followed by the separation processes to prepare the individual fractions, such as rye serum, rye fluid or rye powder. Fractions of rye fluid and rye serum are obtained by centrifugation; a good separation must be achieved by using high-performance centrifuges. Therefore, different centrifuges and additional filtering are employed to ensure high purity. The rye powder is obtained by gentle drying.

### EXAMPLE 2

In the following, some examinations are dealt with in detail.

Changes of the number of peculiarities as compared to the untreated situation and to the starting situation.

Number of Comedones and Inflamed Lesions

The treatment with the test sample resulted in a reduction of the relative number of inflamed lesions and comedones in all subjects at all measuring times (except for subject 1, measuring time 2 weeks). Differences between the areas and changes found on the untreated area, e.g., from climatic changes or individual influences, have been taken into account. Considering all measuring times independently (necessity of performing a statistic consideration in view of the low number of cases) yields a statistically significant reduction (p<0.05) of both the number of inflamed lesions and the number of comedones. The degree of improvement of the inflamed lesions and comedones is relatively high. A tendency with respect to the duration of the application cannot be seen.

Visual Evaluation of the Appearance of the Skin

The visual appearance of the treated side of the face was slightly improved as compared to the untreated side of the face at all measuring times and in all subjects. The optical difference predominantly related to the degree of reddening and the overall impression as well as the size of the individual peculiarities.

Subjective Evaluation by the Subject

All subjects evaluated the effect of the test sample as slightly to clearly positive. In two cases, the subjects reported a reduction of skin reddening. Four subjects felt the treated skin to be smoother than the untreated skin. One female subject described the product as calming to the skin.

During the application phase, three subjects felt a negative sensation in the form of a tension, slight tingling or itching immediately after the application of the product.

Discussion

The test sample rye gel caused a clear improvement of the skin condition of impure skin within the testing period; a complete disappearance of the impression of impure skin could not be observed. The subjects described a positive effect from the test sample.

TABLE 1
Inflamed lesions and comedones

| | | | | |
|---|---|---|---|---|
| Subject | t₀ | 2 weeks | 4 weeks | 6 weeks |

Inflamed lesions

| | | | | |
|---|---|---|---|---|
| 1 | 0 | −8 | −2 | −15 |
| 2 | 0 | 0 | −4 | −3 |
| 3 | 0 | −5 | −6 | −2 |
| 4 | 0 | −7 | −7 | 0 |
| 5 | 0 | −3 | −6 | −7 |

Comedones

| | | | | |
|---|---|---|---|---|
| 1 | 0 | 1 | −1 | −3 |
| 2 | 0 | −6 | −3 | −5 |
| 3 | 0 | 0 | −1 | −1 |
| 4 | 0 | −7 | −1 | −3 |
| 5 | 0 | −2 | −2 | −3 |

TABLE 2
Randomization of the test samples and subject specification

Area (side of the face)

| Subject | Sex | Age (y) | left | right |
|---|---|---|---|---|
| 1 | f | 23.81 | treated | untreated |
| 2 | m | 21.56 | untreated | treated |
| 3 | m | 23.21 | treated | untreated |
| 4 | f | 22.5 | untreated | treated |
| 5 | m | 19.54 | treated | untreated |

Biometry

Target Quantities

The target quantities of the skin care test are skin moisture (corneometry) and skin microtopography (replica, R, DIN).

Evaluation of the Target Quantities

The evaluation of the target quantities was effected by relativizing the original data for the areas treated with the test sample to the untreated situation and the respective starting value (to). The mean value and the standard deviation of the relativized data were determined. The testing of the significance of differences between the untreated and treated situations was effected with the software “STATISTICA”. At first, the distribution of the pair differences of the corresponding pair comparisons was checked. When there was a normal distribution of the pair differences, the significance check was made by means of a two-sided T test for dependent samples. If there had not been a normal distribution of the pair differences, the significance check would have been made with the Wilcoxon test for dependent samples.

Size of the Subject Collective

The size of the subject collective was 15±2.

Discussion

Doubly relativized data, i.e., to the untreated situation and to the starting condition, were evaluated since these take into account the change of the untreated control and the differences in the starting situation between the untreated and the treated areas. From the doubly relativized data, a slight, but statistically significant increase of skin moisture (by about 6%) and a slight, but statistically significant reduction of roughness (by about 7%) as compared to the untreated situation and the starting situation was found after 7 days of treatment with the test sample.

Significant changes with respect to the change of the skin moisture and the skin roughness could not be established after 21 days of treatment and one week of interruption of treatment. The test sample rye gel has a low moisturizing and smoothing effect on the skin.

The climatic conditions in the testing period reflect a warm summerly weather situation with daytime temperatures of about 16° C. to 28° C., much sunshine and a relatively low atmospheric humidity. The first week of application was somewhat cooler as compared to the others, with lesser sunshine and a somewhat increased atmospheric humidity. The climatic conditions on the testing days were different. The day temperatures were approximately equal, the atmospheric humidity varied around about 25%. The measuring climate conditions remained constant during the three testing days.

Due to the randomization of the test samples, all test samples started with the same starting conditions.

Checking of the Inclusion Criteria

All subjects fulfilled the inclusion criteria.

Measuring Results

Skin Moisture

The starting situation is homogeneous, i.e., the untreated areas and the areas to be treated exhibit no significant differences with respect to skin moisture.

At time t₁, the skin moisture increased by an average of about 6% (±12%) due to the test sample treatment as compared to the untreated situation and the starting condition. This difference is statistically significant. After the three weeks of treatment, on average, only a very slight, but non-significant increase (about 2%) of the skin moisture measuring values by the test sample treatment is to be observed. After the one week of treatment interruption, on average, the skin moisture is slightly, but non-significantly increased (about 5%) as compared to the untreated situation and the starting value.

Skin Microtopography

The original data for the determination of skin roughness (replica, R_{z}, DIN) are shown in the annex under “the data relativized to the untreated situation and t₀”.

The starting situation is non-homogeneous, i.e., the untreated areas are significantly smoother than the skin areas to be treated.

At time t₁, the roughness decreased by an average of about 7.5% (+7%) due to the test sample treatment as compared to the untreated situation and the starting condition. This difference is statistically significant. After the three weeks of treatment and the one week of treatment interruption, on average, no reduction of the roughness measuring values from the test sample treatment can be observed any more.

### EXAMPLE 3

In the following, the analysis of the curative and care agent according to the invention is stated.

In preliminary examinations with respect to the separation of a leaven-water suspension by means of centrifugal sedimentation, a three-phase system was established.

In the scope of this examination, it was to be established whether the middle phase is a stable one, of what it could be constituted, and whether an enrichment of important components, such as organic acids, proteins and water-soluble vitamins, occurs therein as compared to the aqueous supernatant. Interpretation indications for a separation of the solids should be attained.

Methods

Centrifugation of the Leaven Suspension

Equipment employed:

Desk-top centrifuge Hermie z 320

Laboratory centrifuge: Hermie z 513

Rheometric Examinations of the Separated Phases

Equipment employed: Rheometer: carri med csl (control stress rheometer)

Method: Establishing of flow-curve with a predetermined shear stress and measured shear rate at a temperature of 23° C.

Chemical Analyses

Sample recovery: In order to obtain sufficient sample for the chemical analyses, 1600 g of leaven mixture was centrifuged at 4300 rpm for 40 minutes. The individual phases were carefully pipetted off and frozen for external analysis.

All chemical analyses were made with the Deutsches Institut für Lebensmitteltechnik. Methods see Results.

TABLE 3
Results of the analysis of the clear aqueous supernatant

| Parameter | Method | Unit | Measuring value |
|---|---|---|---|
| Raw protein | Kjeldahl | g/100 g | 0.73 |
| Acetic acid | HPLC refr. | g/100 g | 0.06 |
| Lactic acid | HPLC refr. | g/100 g | 0.64 |
| Propionic acid | HPLC refr. | g/100 g | <0.01 |
| Butyric acid | HPLC refr. | g/100 g | <0.01 |
| Ascorbic/dehydroascorbic | HPLC | mg/100 g | <5 |
| acid | fluoresc. | | |
| Vitamin B 2 | HPLC-UVD | mg/100 g | <0.5 |
| Vitamin B 6 (pyridoxine) | HPLC-UVD | mg/100 g | <1 |
| Folic acid | HPLC-UVD | mg/100 g | <2 |
| Ethanol | HPLC refr. | g/100 g | 0.07 |

TABLE 4
Results of the analysis of the milky gel-like supernatant
(middle phase)

| Parameter | Method | Unit | Measuring value |
|---|---|---|---|
| Raw protein | Kjeldahl | g/100 g | 0.83 |
| Acetic acid | HPLC refr. | g/100 g | 0.05 |
| Lactic acid | HPLC refr. | g/100 g | 0.64 |
| Propionic acid | HPLC refr. | g/100 g | <0.01 |
| Butyric acid | HPLC refr. | g/100 g | <0.01 |
| Ascorbic/dehydroascorbic | HPLC | mg/100 g | <5 |
| acid | fluoresc. | | |
| Vitamin B 2 | HPLC-UVD | mg/100 g | <0.5 |
| Vitamin B 6 (pyridoxine) | HPLC-UVD | mg/100 g | <1 |
| Folic acid | HPLC-UVD | mg/100 g | <2 |
| Ethanol | HPLC refr. | g/100 g | 0.07 |

### EXAMPLE 4

The cosmetic preparation according to the invention is suitable for use with the following phenomena.

Acne:

In the initial phase, fermented raw material was applied fresh (live cultures) to the facial skin and removed with warm water after the “mask” had dried.

Alternatively, a pasteurized “mask” may also be used.

The results were evaluated as being nearly identical by the subjects which had applied both products:

Rosy soft skin well supplied with blood; the acne picture was highly improved. In two cases, total healing was observed. However, after 3 days, the formation of acne pustules started again, but these could then again be treated with a fermentation regeneration mask according to the invention.

With acne scars, substantial improvements could be observed already after three treatments. The skin had a softer look and made an impression of care and of being well supplied with blood (treatment see above).

Scalp:

Here, the cosmetic preparation according to the invention in the form of the serum proved suitable for different problems.

A typical application is as follows:

Before being washed, the hair is parted, and the product is incorporated by a strokewise massage. After a reaction time of 15 minutes, the hair is washed with a mild shampoo as usual.

Scales, inflamed parts up to partial hair loss after detaching of major scalp zones could be improved and in part even healed.

Results

Deviations from the Test Schedule

There were no deviations from the test schedule.

Undesirable events: 1 cancellation

There were no unforeseeable events. Five subjects went through the test correctly and completely.

Test of the Inclusion Criteria

All subjects met the inclusion criteria.

Application Times of the Test Samples

The application times of the test samples met the requirements.

Results of Visual Evaluation

The tolerability of 6 test samples, neat and in 50% dilution, was examined on the basis of one application of the test samples. A test sample was rated “peculiar” if the result of visual evaluation was ≧1.0, i.e., at least a slight spot-like or diffuse reddening was observed.

The test samples wheat serum, wheat gel, oat gel (neat and 50%) and the test samples oat serum and rye gel (neat) were not peculiar with any of the subjects.

To the test samples oat serum and rye gel in 50% dilution and the test sample rye serum (neat and 50%), one subject each (20% of all subjects) reacted with a slight spot-like or diffuse reddening. The maximum of the reaction to these test samples occurred 24 h after the end of the exposition. In all cases, the reactions were restricted to the application area.

1. A method for the preparation of a curative and care agent for the skin on the basis of an acidified leaven, wherein:
1.1 a natural enzymatic starter and water are added to a rye fine or coarse meal, the mixture is subjected to a process of heating to 65-75° C. within 3 to 5 hours, a strong maltose formation being initiated from enzymatic reactions;
1.2 a further addition of rye fine or coarse meal, water and a bacteriological inoculum from the group of heterofermentative lactic-acid bacteria is made; and
1.3 the mixture is acidified until the metabolic activity of the microorganisms ceases.

2. The method according to claim 1, wherein said enzymatic starter is α-amylase.

3. The method according to claim 1, wherein said heterofermentative lactic-acid bacteria are Lactobacillus DSM 6037 and/or Lactobacillus DSM 6129.

4. The method according to claim 1, wherein a second carbohydrate-containing substrate and water as well as a bacteriological inoculum from the group of heterofermentative lactic-acid bacteria are added to the fermentation product, and the mixture is acidified;
the latter process step is optionally repeated once or several times, and the mixture is finally acidified until any bacteriological metabolic activity ceases; and
the fermentation product is subjected to a stabilizing heating process at near to the boiling temperature.

5. A curative and care agent for the skin on the basis of an acidified leaven obtainable by the method according to claim 1.

6. The curative and care agent according to claim 5, characterized in that said curative and care agent has a high content of lactic acid obtained in a fermentative way of >60% (as a fraction of the total amount of acid), wherein a treatment with α-amylase is performed prior to acidification of the leaven.

7. The curative and care agent according to claim 5 comprising auxiliary agents and carriers usual in the cosmetic art.

8. The curative and care agent according to claim 5 in the form of powders, rye fluids, rye sera, rye gels.

9. A cosmetic preparation containing a curative and care agent according to claim 5 in the form of ointments, creams, lotions, gels, shampoos, hair conditioners, packs, lipsticks, face and body masks, or combinations thereof.

10. Use of a curative and care agent according to claim 5 in a cosmetic preparation in the form of ointments, creams, lotions, gels, shampoos, hair conditioners, packs, lipsticks, face and body masks, or combinations thereof.
